(19) United States
(12) Patent Application Publication
(10) Pub. No.: US 2003/0088300 A1
(43) Pub. Date: May 8, 2003

(54) "Thermangel" external body temperature regulator

(76) Inventor: Mary Frances Vester, Nashville, TN (US)

Correspondence Address: Mary Frances Vester, 1205 Plymouth Avenue, Nashville, TN, 37216, US

(21) Appl. No.: 09/994,000

(22) Filed: Nov. 6, 2001

(51) Int. Cl. A61F 7/00

(52) U.S. Cl. 607/109; 607/114

(57) ABSTRACT

The Thermangel thermoregulating device is a unique approach to treating hyperthermia/hypothermia and other serious conditions. By the process of regulating the temperature around the hypothalamus, the regulatory center of the body, we can effect the ability of the body to regulate itself. This process allows the human body to return to normal or homeostatic conditions. The process of hypothermia and hyperthermia is currently being studied as a treatment for conditions such as alopecia, hair loss due to chemotherapy. As well as improving the survival of cardiac arrest resuscitations. It is essentially non-invasive and will decrease medical costs for everyone. It decreases the amount of time spent in the critical-care setting, decreases the need for medications and intravenous fluids and provides comfort to the patient. This is also a new and effective way to treat Malignant Hyperthermia, which is a deadly condition found the surgical environment. It will provide a cost-effective solution to improve heath care for the patient, hospital and related entities. In the future, this product could be furthered improved by placing an electrical heating/cooling unit to set the exact temperature desired.

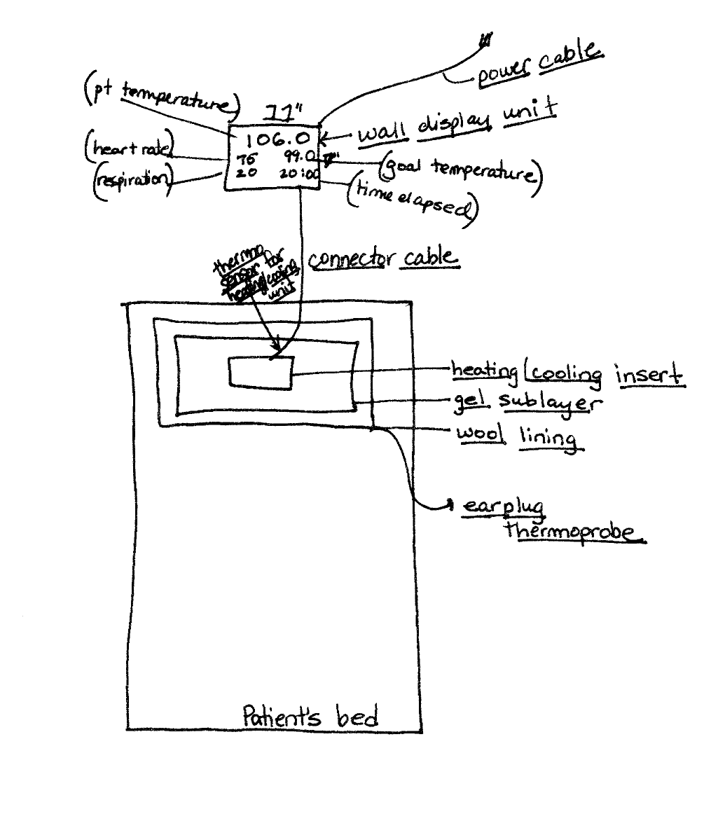

### BACKGROUND OF THE INVENTION

This invention was designed on Oct. 3, 2001. Its conception began over 5 years ago, while working as a nurse in a community hospital. I was taking care of a patient whose temperature began to climb above 104 degrees. In trying to bring down the patient's temperature, I had a moment of serendipity. If the cooling center of the body is in the hypothalamus, located at the base of the brain, why not put some ice in a bag and put it under the head? So I got some ice bags and put them their. Well, as you may have already assumed, it worked, and it worked beautifully. The patient's temperature decreased to 99.0 F in approximately 4 hours. And that is where it began. After taking care of many critically-ill patients, especially those with sepsis, this idea became my mainstay. It was so much easier than previously used methods and did not make near as much mess. Much expense both financial and otherwise can be attributed to thermoregulation difficulties.

### DESCRIPTION OF THE PRIOR ART

To describe the prior art of thermoregulation intervention is two part. I will describe how hyperthermia/hypothermia was previously treated as well as the closest cousin to this design.

Hyperthermia is a condition where the temperature is too high. This is commonly found in sepsis, a massive infection state where the body begins to shut down from the illness. Hyperthermia is also found in drug reactions, heatstroke, thyroid disorders, alcohol withdrawal and many other illnesses. It is commonly treated with ice baths, cooling blankets and ice packs to the groin and axilla (underarms). These approaches are both problematic, time-consuming and less than effective at times. Usually when someone is hyperthermic, they are critically ill and near death.

Hypothermia is a condition where the temperature of the body is too low. This happens due to exposure of the elements, near drownings and accidental exposures. The current techniques used to treat this are gastric lavage (heated water flushed though the stomach to heat the body core) and also peritoneal lavage (flushing heat water into the peritoneal cavity). It is the preferred method to heat from the body core outward as opposed to heat the extremities. It is the idea to prevent heated blood from going to the major organs. So the technology today is to heat from inside out and not outside in. This technology follows the same approach. It is heating from the center of the body outward.

The closest designs I could find were used for other purposes. For example, the Chillow is a flat, thin product placed in between the pillow and the pillowcase to keep your pillow cool and treat minor heat related problems such as sunburn and backache. It is not large enough to treat this condition. Also, it was not designed with medical use in mind. There is also a product that heats from the palm of the hand. There also various cooling devices used for coolers that are small, blue ice packs. You may have one in your home. My device is different in that it reduces or raises the body temperature and then maintains it. You must be very careful in that you do not lower the temperature too much and that the achieved temperature is maintained.

### SUMMARY OF THE INVENTION

The Thermangel is a three piece, vinyl polymer unit that resembles a pillow. The patient lays on the pillow and is thermoregulated by lying on the pillow by way of cooling/heating the base of the skull. The pillow has three compartments with a wool lining, gel layer and insert layer. The pillow has an attached probe on the right side with an earplug to monitor the patient's temperature. There is also a cord at the top of the pillow connecting to the display unit. There is also a cord on the back of the wall unit connecting to the power source, which is the standard electric outlet found in all rooms. The thermal insert unit is premeasure to treat specific temperatures from 109 to 85 degrees Fahrenheit. Ice is placed in the insert containers for cooling and microwave heated water is placed in the heating insert. They are premeasured so that you are not provided to much heat or cold to a patient.

### BRIEF DESCRIPTION OF THE DRAWINGS

see attached folder

### DETAILED DESCRIPTION OF THE DRAWING

The thermal pillow has a hollow wool lining. Wool has been long used for its insulative qualities for both heat and cold. The next layer is a square donut-shaped gel layer. The pillow and its subcomponents are all made of a vinyl polymer, similar to the inside of a flexible cooler or lunchbag. You could imagine the inside of a rubber galosh. The next layer, composed of the vinyl polymer, is filled with a non-toxic gel layer that is shaped like a square-shaped donut. The inner layer is a compartment for the heating or cooling device. The heating insert is also a vinyl polymer with screw on cap to prevent leakage. The cooling insert is also a vinyl polymer with screw on cap. There is a seam along the middle of the pillow for inserting the desired heating/cooling application. On the right side of the pillow is a cord, approximately 24 inches long, which connects to an earprobe to regulate the temperature of the patient. There is also two more cords located at the top section of the pillow. One connects to the electric outlet and the other connects to a digital wall monitor. The monitor shows the patients current temperature, the temperature of the heating/cooling unit, how long the patient has been on this therapy, and how many degrees of difference. In addition, we would like to add the patient's heart rate and rate of respirations per minute.

1. The Thermangel would be suited for thermocorrecting the human body. It has the ability to cool the human body, approximately 180 pounds, approximately 1-2 degrees per hour. The patient in a hyperthermic state, usually has a corresponding tachycardia, or increased heart rate. If the heart rate goes too high, it can impair the function of the heart. For example, one patient had a temperature of 106.1 and a heart rate of 138. The heart rate should normally be 60-100. After being cooled for 6 hours using this process, the heart rate had decreased to 104 with a temperature of 99.0 F. Normal body temperature is approximately 98.6 degrees. Some people vary and may be lower or higher. Prolonged hyperthermia causes dehydration, confusion, electrolyte disturbances and in some cases, seizures and death. This device has an excellent ability to return the body to homeostasis or normal conditions. This will also help decrease medical costs due to a decreased length of stay in the hospital in addition to improve patient outcomes. When people have a high temperature, 104 or greater usually, they are delirious and confused if not comatose. Sometimes these patients are agitated and have to be restrained. Returning the temperature to normal would improve the current standard of treatment for these people. This unit will not cure why the patients have a temperature, but it will effectively eliminate one of the most problematic symptoms of a septic patient.
The cooling apparatus warms the body approximately 1-2 degrees per hour. Frequently, people who have had near drowning accidents are hypothermic. They must have their core temperature returned to normal to prevent complications such as cardiac arrest and brain damage. This device would also be helpful during surgery to keep the patient's temperature from getting too high, a condition known as Malignant Hyperthermia. This is a condition triggered by a rare reaction to certain anesthetic medications. It becomes a medical emergency to reduce the patients temperature, as this condition can be the cause of death for many who have this type of reaction.
This unit would also be useful for therapeutic hyperthermia, which is currently being researched as an adjunctive therapy for cancer patients. It also allows the patient to time the amount of therapy as well as target the correct prescribed temperature. It would allow the cancer patients to do the therapy at home instead of in the hospital. Research so far has shown it to have beneficial effects for patients receiving radiation and hyperthermic treatments from a rate of 33% for radiation alone to 67% for the patients that used both treatments. It is also helpful in reducing alopecia, hair loss, for patients receiving chemotherapy treatments.
It would also be very useful for infants and children who are febrile, or running a fever.
Children dehydrate very quickly from running a temperature. Instead of a parent waking up and taking the child's temperature throughout the night, she or he would be able to look at the monitor and assess the child's response without waking them to take a temperature. Also, a cold surface feels good to a child that is febrile. I can remember many times when my little girl would lay her face on the kitchen floor because she was running a fever and the cold floor felt good to her.
In addition to thermoregulating temperature, this system could be used in future research to combat cardiac and respiratory conditions. As this unit is placed at the base of the brain, under the hypothalamus. The hypothalamus control many of the body's functions such as heart rate and respiration or breathing. I would also recommend for study its usefulness for anxiety and migraines as well as cooling athletes following physical exertion. They have previously used cooling tubs, which are expensive and inconvenient. Instead, they could just go lay down, take a nap and monitor their temperature. This unit would also be helpful couples suffering with infertility who must take daily temperatures, instead they could just put in the earplug and lay down, instead of inserting a vaginal or rectal thermometer.
The overwhelming advantage to this unit is that it is non-invasive. You do not have to stick someone with a needle to use this. Some of the most recent developments in temperature regulation involve opening the human body. For example, peritoneal lavage to decrease body temperature. This involves inserting warmed water or saline into the abdominal cavity. Invasive techniques always involve the risks of infection, bleeding and stroke. This therapy requires very little monitoring. In light of the current nursing shortage and increasing elderly population, this would foster maximum clinical efficacy with minimal monitoring required. We are currently investigating adding the ability to display the patient's heart rate on the display screen as well. This would be ideal for patients who are not monitored using sophisticated equipment in the telemetry and intensive care unit. So you are increasing the ability to monitor patients without additional cost to the patient, hospital or managed care organization. It would also help to cut medical costs for everyone as illness can be managed with fewer medications needed. Current research in medicine has shown that resuscitation after cardiac arrested is improved if cooling techniques are utilized.
